(19) United States
(12) Patent Application Publication
(10) Pub. No.: US 2001/0014340 A1
(43) Pub. Date: Aug. 16, 2001

(54) INTRABUCCALLY RAPIDLY DISINTEGRATING TABLET

(76) Inventors: MOTOHIRO OHTA, SHIZUOKA (JP); EIJI HAYAKAWA, SHIZUOKA (JP); KUNIO ITO, SHIZUOKA (JP); SANJI TOKUNO, TOKYO (JP); KIYOSHI MORIMOTO, SHIZUOKA (JP); YASUSHI WATANABE, SHIZUOKA (JP)

Correspondence Address: FELIX J D'AMBROSIO, JONES TULLAR & COOPER, PO BOX 2266 EADS STATION, ARLINGTON, VA, 22202

(21) Appl. No.: 09/147,374

(22) PCT Filed: Jun. 12, 1997
(86) PCT No.: PCT/JP97/02032

§ 371 Date: None

(30) Foreign Application Priority Data
Jun. 14, 1996 (JP) 8-153553
Mar. 25, 1997 (JP) 9-71107

(51) Int. Cl. A61K 9/00

(52) U.S. Cl. 424/400

(57) ABSTRACT

A tablet comprising sugar alcohol or saccaride having an average particle diameter of not more than 30 μm, an active ingredient, and a disintegrant. It is an intraorally rapidly disintegrable tablet which does not require a special pharmaceutical manufacturing technology and can be simply and easily produced by a normal equipment.

### TECHNICAL FIELD

This invention relates to a tablet rapidly disintegrable in an oral cavity.

### BACKGROUND ART

There are various types of oral administrative medicines: tables, capsules, granules, powders, syrups and so on. However, such oral administrative medicines have several problems as follows. As to tablets and capsules, for example, it may be hard for aged persons or children whose swallowing power is weak to swallow them. And as to granules and powders, they may cause unpleasantness in a mouth after dosage or they may erroneously happen to enter into a respiratory tract or lungs. Further, they can not be taken where there is no water because water is usually required for dosage. As for syrups, it takes trouble for measuring on dosage and it is not be expected that aged persons or children measure them accurately. On the other hand, solid medicines which can be rapidly dissolved or disintegrated in an oral cavity can be taken without measuring or water, so they may be easily taken by such aged persons or children. By the way, there have been developed several types of medicines which can be rapidly dissolved or disintegrated in an oral cavity on dosing. For instance, in JP-B-62-50445, solid medicines which can be produced from water solution that mainly contains gelatin including an active ingredient by use of freeze drying method are disclosed. And in WO93/12769, solid medicines which can be produced by drying suspension including agar are also disclosed. However, the medicines produced by the above-mentioned prior method do not have enough hardness to be taken out by pushing from PTP packages (Press Through Pack) containing the medicines. And, they require a special pharmaceutical manufacturing technology and also require an enormous investment in plants and equipments. JP-A-5-271054 and WO93/15724 disclose production methods of tables wherein tablets composed of saccaride are produced in such a manner that saccaride mixture supplied with appropriate water is compressed at a low pressure and then dried to make solid tablets. However, such methods also require a special pharmaceutical manufacturing technology and have the fear that powder composed of the tablets may adhere to the surface of a metal mold in compression process under moistening condition. It may be therefore difficult to utilize those methods for manufacturing on an industrial scale.

### DISCLOSURE OF THE INVENTION

The inventors of the present invention have examined an intraorally rapidly disintegrable tablet which does not require a special pharmaceutical manufacturing technology and can be simply and easily produced by a normal equipment. As a result, they have found that the compressed tablet consisting essentially of sugar alcohol or saccaride, such as D-mannitol or lactose, having an average particle diameter of not more than 30 μm as a main ingredient, an active ingredient and a disintegrant can be disintegrated in a mouth within one minute and have hardness adequate for practical use, although such tablet has been considered unable to produce for a long time.

Accordingly, the present invention relates to a tablet comprising sugar alcohol or saccaride having an average particle diameter of not more than 30 μm, an active ingredient and a disintegrant.

Further the present invention relates to a production method of a tablet characterized by compressing powdered mixture which comprises sugar alcohol or saccaride each having an average particle diameter of not more than 30 μm, an active ingredient, and a disintegrant.

In the present invention, such as D-mannitol, solbitol, or the like, which is widely used for drugs and food, can be used as sugar alcohol, and lactose and glucose or the like, which is also widely used for drugs and food, can be used as saccaride. At least one kind of sugar alcohol or saccaride is used.

As an active ingredient, followings are used, but other ingredients for oral administration can be also used.

<drug for central nervous system>

hyprotic, anxiolytic . . . amobarbital, alprazolam, flurazepam hydrochloride, diazepam, and so on

antiepileptic . . . sodium valproate, nitrazepam, phenytoin, and so on

NSAID/analgesic antipyretic agent . . . aspirin, acetaminophen, ibuprofen, diclofenac sodium, ethenzamide, indometacin and so on

drug for Parkinson's disease . . . levodopa, amantadine hydrochloride, trihexyphenidyl hydrochloride, piroheptine hydrochloride, and so on

psychoneurosis agent . . . etizolam, amitriptyline hydrochloride, sulpiride, and so on

<drug for peripheral nervous system>

skeletal muscle relaxant . . . chlorphenesin carbamate, chlormezanone, and so on

autonomic nervous agent . . . valethamate bromide, tofisopam, and so on

antispasmodic . . . afloqualone, and so on

<drug for circulatory organ>

cardiac . . . ubidecarenon, aminophylline, etilefrine hydrochloride, and so on

antiarrhythmic agent . . . atenolol, pindolol, and so on

diuretic . . . spironolactone, trichlormethiazide, furosemide, and so on

antihypertensive agent . . . todrazine hydrochloride, nicardipine hydrochloride, hydralazine hydrochloride, and so on

angiotonic . . . dihydroergotamine mesilate and so on

vasodilator . . . benidipine hydrochloride, diltiazem hydrochloride, isosorbide dinitrate, and so on

hyperlipemia . . . clinofibrate, nicomol, and so on

others . . . flunarizine hydrochloride, meclofenoxate hydrochloride, cinnarizine, and so on

<alimesitary tract drug>

antidiarrheal drug . . . loperamide hydrochloride, dimeticone, and so on

drug for peptic ulcer . . . azulene, L-glutamine, aceglutamide aluminium, cetraxate hydrochloride, cimetidine, and so on

cholagogue . . . anetholtrithion, chenodeoxycholic acid, and so on

others . . . domperidone, trimebutine maleate, metoclopramide, cisapride and so on

<metabolic drug>

vitamin . . . alfacarcidol, tiamine hydrochloride, cobamide, vitaroxin riboflavin butyrate, ascorbic acid, phytonadione, and so on

diabetes mellitus agent . . . glybuzole, tolbutamide, and so on

<antiallergics>

antihistamine . . . homochlorcyclizine hydrochloride, clemastine fumarate, chlorpheniramine maleate, and so on

others . . . oxatomide, ketotifen fumarate, azelastin hydrochloride, and so on

<antineoplastics>

antimetabolite . . . fluorouracil, tegafur, and so on

<antibiotics>

paromomycin sulfate, amoxicillin, cefaclor, cefalexin, acetylspiramycin, minocycline hydrochloride, and so on

In the present invention, crosspovidone, crosscarmellose sodium, low substituted hydroxypropylcellulose or the like, which are widely used for drugs and food can be used as a disintegrant. At least one kind of disintegrant is used.

Next, a production method of a tablet according to the present invention will be described hereinafter.

The tablet of the present invention can be obtained by compressing and tableting after granulating a mixed powdered component comprising sugar alcohol or saccaride having an average particle diameter of not more than 30 μm ground by means of a hammer mill or a jet mill or the like, an active ingredient, and a disintegrant. On the other hand, the tablet of the present invention also can be obtained by compressing and tableting after granulating a mixed powdered component comprising sugar alcohol or saccaride having an average particle diameter of not more than 30 μm ground by means of a hammer mill, a jet mill or the like, an active ingredient, and a disintegrant under the presence of an easily volatile disintegrating adjuvant and thereafter the disintegrant adjuvant is volatilized.

The amount of sugar alcohol or saccaride is preferably about 60˜95%, more preferably about 80˜95% per one tablet.

The amount of active ingredient is different depending on the kind and dosage amount of active ingredients, however, 0.01˜30% is preferable, and more preferably 0.01˜10% per one tablet.

The amount of disintegrant present is preferably about 1˜30 mg per dosage, and more preferably 1˜10% per one tablet.

Easily volatile disintegrating adjuvant is such as sublimative camphor, urethane, urea, ammonium bicarbonate, benzonic acid, or the like, however, camphor is most preferable. The amount of easily volatile disintegrating adjuvant is preferably 1˜20% and more preferably 1˜10% per one tablet.

A wet granulation method using purified water, ethanol or the like can be preferably used. In the method, for example, granulation can be executed by means of a general granulator such as a fluid-bed granulator, a rotary stirring granulator or an extruding granulator. The granulated material is dried, and mixed with a lubricant, and thereafter compressed into predetermined shape. Binder, sour agent, foaming agent, sweetening agent, flavoring agent, or colorant can be added as additive. As a lubricant, such as magnesium stearate, stearic aid, stearyl alcohol, sucrose ester of fatty acid, talc, light anhydrous silicic acid, or like can be used. Binder is, for example, hydroxypropylcellulose, polyvinylpyrrolidone, hydroxypropylmethylcellulose, partially saponificated polyvinyl alcohol methylcellulose, pullulan or the like. Sour agent is citric acid, malic acid, adipic acid, ascorbic acid, or the like. Foaming agent is sodium bicarbonate, sodium carbonate, calcium carbonate, or the like. Sweetening agent is Aspartame (TM), saccharin, glycyrrhizic acid or the like. Flavoring agent is lemon, orange, pine, mint, menthol or the like. Colorant is yellow iron sesquioxide, red iron sesquioxide, tar color or the like. The amount of lubricant is preferably 0.01˜1% and more preferably 0.01˜0.5% per one tablet.

Although a method of compression is not limited in the present invention, a rotary tablet machine, a hydraulic press machine or a single punch tableting machine which have high productivity can be more preferably used. When an easily volatile disintegrating adjuvant is used, the tablet is dried by heating after compressing process. As to lubricants, they can be excluded from the powdered mixture in the granulation process, in that occasion it may be previously spread on the surfaces of punches and dies of a tableting machine before compression process. That makes the present invention more effective. Compression pressure of a rotary tablet machine may be preferably more than 300 kg.

The shape of the tablet obtained in the present invention can be pills or other shapes such as a normal R surface tablet, a sugar coated R surface tablet, a tablet with square edges, a tablet with rounded edges, or a tablet with two R surfaces, or the like.

Further, the tablet may have a divisional line.

### BEST MODE FOR CARRYING OUT THE INVENTION

The present invention will be concretely explained according to examples and reference examples.

### REFERENCE EXAMPLE 1

1890 g of D-mannitol (Towa Kasei Co., Ltd. : average particle diameter of about 60 μm) and 10 g of crosspovidone (POLYPLASDONE XL-10: GAF Co., Ltd.) were fed in a fluid-bed granulation dryer (Glatt Co., Ltd.: WSG-type 5), purified water was sprayed, then granulated material was obtained after granulation and drying processes. 10 g of magnesium stearate was added and mixed with the granulated material, and they were compressed and tableted by a rotary tablet machine (Kikusui Seiko Co., Ltd., CLEAN PRESS COLLECT TYPE 12). Tableting conditions were as follows; tablet weight was 200 mg, a metal mold was 8 mm diameter flat-type, and compression pressure was varied such as 150 kg, 300 kg, 450 kg, 600 kg, and 800 kg.

### Example 1

D-mannitol (Towa Kasei Co., Ltd.: average particle diameter of about 60 μm) was previously ground by a jet mill (Japan Pneumatic Co., Ltd.:type PJM-1-1.5) and the pulverized D-mannitol with average particle diameter of 20 μm was obtained. 1890 g of the pulverized D-mannitol and 100 g of crosspovidone (POLYPLASDONE XL-10:GAF Co., Ltd.) was fed in a fluid-bed granulation dryer (Glatt Co., Ltd. : WSG-type 5), purified water was sprayed, and granulated material was obtained after granulation and drying processes. 10 g of magnesium stearate was added and mixed with the granulated material, and they were compressed and tableted by a rotary tablet machine (Kikusui Seiko Co., Ltd., CLEAN PRESS COLLECT TYPE 12). Tableting conditions were the same as the reference example 1.

### REFERENCE EXAMPLE 2

100 g of domperidone, antiemetic agent, 1790 g of lactose (DMV Co., Ltd. : average particle diameter of about 80 μm) and 100 g of crosspovidone (POLYPLASDONE XL-10:GAF Co., Ltd.) were fed in a fluid-bed granulation dryer (Glatt Co., Ltd. : WSG-type 5), purified water was sprayed, and granulated material was obtained after granulation and drying processes. 10 g of magnesium stearate was added and mixed with the granulated material, and they were compressed and tableted by a rotary tablet machine (Kikusui Seiko Co., Ltd.,: CLEAN PRESS COLLECT type 12). Tableting conditions were the same as the reference example 1.

### Example 2

Lactose (DMV Co., Ltd.: average particle diameter of about 80 μm) was previously ground by a jet mill (Japan Pneumatic Co., Ltd.: type PJM-1-1.5) and the pulverized lactose having average particle diameter of 15 μm was obtained. 1790 g of the pulverized lactose, 100 g of domperidone, and 100 g of crosspovidone (POLYPLASDONE XL-10:GAF Co., Ltd.) were fed in a fluid-bed granulation dryer (Glatt Co., Ltd.: WSG-type 5), purified water was sprayed, and granulated material was obtained after granulation and drying processes. 10 g of magnesium stearate was added and mixed with the granulated material, and they were compressed and tableted by a rotary tablet machine (Kikusui Seiko Co., Ltd., CLEAN PRESS COLLECT TYPE 12). Tableting conditions were the same as the reference example 1.

### Example 3

The granulated material obtained in the example 1 was tableted under the condition that tablet weight was 200 mg, compression pressure was 50 kg/c m², and a little magnesium stearate was coated on a metal mold (8 mm diameter flat-type) and dies of a hydraulic press machine (Riken Seiki Co., Ltd.: type P-1B)

### Example 4

140 g of the pulverized D-mannitol used in the example 1, 10 g of domperidone, 10 g of crosspovidone (POLYPLASDONE XL-10:GAF Co., Ltd.) and 40 g of camphor were mixed in a vinyl bag and tableted under the condition that tablet weight was 200 mg, a metal mold diameter was 9 mm, a single punch tableting machine (Okada Seiko Co., Ltd.: N-20E type both pressure powder tableting machine) was used, and compression pressure was 1500 kg/cm². The compressed tablet was dried for 10 minutes at 80° C. under vacuum condition in a vacuum dryer.

Next, the hardness and disintegrating time of the tablet of the present invention will be described using an experimental example.

### Experimental Example

The hardness and the disintegrating time of the tablet obtained by the examples 1 and 2 and reference examples 1 and 2 were measured. The hardness of the tablet was measured by a tablet destructive strength measuring instrument (Toyama Sangyo Co., Ltd. : TH-203CP type) The disintegrating time of the tablet was measured in such a way that the tablet was placed on a No. 10 wire cloth, water was dropped at a speed of 4 ml/min. on the tablet, and the time till the tablet go through the wire cloth was measured. The time was determined as the disintegrating time.

The result is shown in a Table 1.

TABLE 1

| sample/compression pressure | | 150 kg | 300 kg | 450 kg | 600 kg | 800 kg |
|---|---|---|---|---|---|---|
| comparison 1 | hardness | hard to be | hard to be | hard to be | 1.9 kgf | 2.3 kgf |
| | | tableted | tableted | tableted | | |
| | disintegration | — | — | — | 20 sec. | 22 sec. |
| embodiment 1 | hardness | 1.9 kgf | 3.9 kgf | 5.1 kgf | 6.2 kgf | 7.3 kgf |
| | disintegration | l0 sec. | 15 sec. | l6 sec. | l9 sec. | 27 sec. |
| comparison 2 | hardness | hard to be | hard to be | hard to be | 1.5 kgf | 2.1 kgf |
| | | tableted | tableted | tableted | | |
| | disintegration | — | — | — | l8 sec. | 2l sec. |
| embodiment 2 | hardness | 1.6 kgf | 4.0 kgf | 4.9 kgf | 5.8 kgf | 6.5 kgf |
| | disintegration | l0 sec. | l6 sec. | 20 sec. | 25 sec. | 29 sec. |

In reference examples 1 and 2, tableting was hard till 450 kg compression pressure and tableting was made possible at around 600 kg. However, the hardness of the tablet was not enough. In examples 1 and 2, enough tablet hardness could be obtained at more than 300 kg compression pressure and the disintegrating time was very fast. When the tablet produced at 450 kg compression pressure according to the example 1 was dosed, the tablet was disintegrated within 10 seconds in an oral cavity.

The tablets obtained by the examples 3 and 4 were also measured of its hardness and disintegrating time in the same way. The tablets obtained by the example 3 had enough hardness of about 6.5 kgf and its disintegrating time was about 10 seconds. The hardness of the tablet of the example 4 was about 4 kgf and its disintegrating time was about 2 seconds, which was very fast.

### Industrial Applicability

According to the present invention, a tablet rapidly disintegrable in an oral cavity can be provided.

1. A tablet comprising;
sugar alcohol or saccaride each having an average particle diameter of not more than 30 μm,
an active ingredient, and
a disintegrant.

2. The tablet as set forth in claim 1, wherein the tablet contains disintegrant of 1 to 30 mg for one dosage.

3. The tablet as set forth in claim 1 or 2, wherein said sugar alcohol is D-mannitol.

4. The tablet as set forth in claim 1 or 2, wherein said saccaride is lactose.

5. The tablet as set forth in claim 1, 2, 3 or 4, wherein said disintegrant is crosspovidone, crosscarmellose sodium, or low substituted hydroxypropycellulose.

6. A production method of a tablet characterized by compressing powdered mixture which comprises sugar alcohol or saccaride each having an average particle diameter of not more than 30 μm, an active ingredient, and a disintegrant.

7. The production method of a tablet as set forth in claim 6, wherein a tableting machine is employed in compressing process of the method which is provided with punches and dies previously spread with lubricants.

8. The production method of a tablet as set forth in claim 6, wherein powdered mixture which comprises sugar alcohol or saccaride each having an disintegrant is compressed into the tablet under the existence of a readily volatilizable disintegrating adjuvant and thereafter the disintegrant adjuvant is volatilized from the compressed tablet.
